(19) United States
(12) Patent Application Publication
(10) Pub. No.: US 2024/0095907 A1
(43) Pub. Date: Mar. 21, 2024

(54) DEEP LEARNING BASED SYSTEMS AND METHODS FOR DETECTING BREAST DISEASES

(71) Applicant: United Imaging Intelligence (Beijing) Co., Ltd., Beijing (CN)

(72) Inventors: Zhang Chen, Brookline, MA (US); Shanhui Sun, Lexington, MA (US); Xiao Chen, Lexington, MA (US); Yikang Liu, Cambridge, MA (US); Terrence Chen, Lexington, MA (US)

(73) Assignee: United Imaging Intelligence (Beijing) Co., Ltd., Beijing (CN)

(21) Appl. No.: 17/948,726

(22) Filed: Sep. 20, 2022

(51) Int. Cl. G06T 7/00; G16H 30/40; G16H 50/20

(52) U.S. Cl. CPC G06T 7/0012; G16H 30/40; G16H 50/20; G06T 2207/10112; G06T 2207/20081; G06T 2207/20084; G06T 2207/30068

(57) ABSTRACT

Mammography data such as DBT and/or FFDM images may be processed using deep learning based techniques, but labeled training data that may facilitate the learning may be difficult to obtained. Described herein are systems, methods, and instrumentalities associated with automatically generating and/or augmenting labeled mammography training data, and training a deep learn model based on the auto-generated/augmented data for detecting a breast disease (e.g., breast cancer) in a mammography image.

### BACKGROUND

Breast cancer is a common cause of death among women in all parts of the world, accounting for a large part of new cancer cases and hundreds of thousands of deaths each year. Early screening and detection are key to improving the outcome of breast cancer treatment, and can be accomplished through mammography exams (mammograms). Newer generations of mammogram technologies can provide much richer information for disease diagnosis and prevention, but the amount of data generated by these technologies may also increase drastically, making image reading and analysis a daunting task for radiologists. To reduce the workload of the radiologists, machine learning (ML) such as deep learning based techniques have been proposed to process mammograph images using pre-trained ML models. The training of these models, however, requires a large number of manually annotated medical images, which are difficult to obtain.

### SUMMARY

Described herein are deep learning based systems, methods, and instrumentalities associated with processing mammography images such as digital breast tomosynthesis (DBT) and/or full-field digital mammography (FFDM) images. An apparatus capable of performing such tasks may include at least one processor that may be configured to obtain a medical image of a breast, determine, based on the medical image, whether an abnormality exists in the breast, and indicate a result of the determination (e.g., by drawing a bounding box around the abnormality). The determination may be made based on a machine-learned abnormality detection model that may be learned through a process that comprises: training an abnormality labeling model based on a first training dataset comprising labeled medical images; deriving a second training dataset based on unlabeled medical images, wherein the derivation may comprise annotating the unlabeled medical images based on the trained abnormality labeling model; and training the abnormality detection model based at least on the second training dataset. The abnormality detection model thus obtained may be a different model than the abnormality labeling model or a refinement of the abnormality labeling model.

The abnormality labeling model described herein may be trained to predict an abnormal area in an unlabeled medical image and annotate the unlabeled medical images based on the prediction. Such annotation may comprise marking the predicted abnormal area in the each of the unlabeled medical images, for example, by drawing a bounding box around the predicted abnormal area. In examples, the derivation of the second training dataset may further comprise transforming at least one of an intensity or a geometry of the medical images annotated by the abnormality labeling model, and/or transforming at least one of an intensity or a geometry of a markup (e.g., a bounding box) created by the labeling model. In examples, the derivation of the second training dataset may further comprise masking one or more medical images annotated by the abnormality labeling model, and/or removing a redundant prediction of an abnormal area in the medical images annotated by the abnormality labeling model. In examples, the derivation of the second training dataset may further comprise determining that a medical image annotated by the abnormality labeling model may be associated with a confidence score that is below a threshold value, and excluding such medical image from the second training dataset based on the determination.

### BRIEF DESCRIPTION OF THE DRAWINGS

A more detailed understanding of the examples disclosed herein may be had from the following description, given by way of example in conjunction with the accompanying drawings.

FIG. 1A and FIG. 1B are simplified diagrams illustrating examples of mammography techniques, according to some embodiments described herein.

FIG. 2 is a simplified diagram illustrating an example of using machine learning (ML) techniques to automatically detect abnormalities in mammogram images, according to some embodiments described herein.

FIG. 3 is a simplified diagrams illustrating example operations that may be associated with training a neural network or ML model for processing mammogram images, according to some embodiments described herein.

FIG. 4 is a flow diagram illustrating an example method for training a neural network to perform one or more of the tasks as described with respect to some embodiments provided herein.

FIG. 5 is a simplified block diagram illustrating an example system or apparatus for performing one or more of the tasks as described with respect to some embodiments provided herein.

### DETAILED DESCRIPTION

The present disclosure is illustrated by way of example, and not by way of limitation, in the figures of the accompanying drawings. A detailed description of illustrative embodiments will now be described with reference to the various figures. Although this description provides a detailed example of possible implementations, it should be noted that the details are intended to be exemplary and in no way limit the scope of the application.

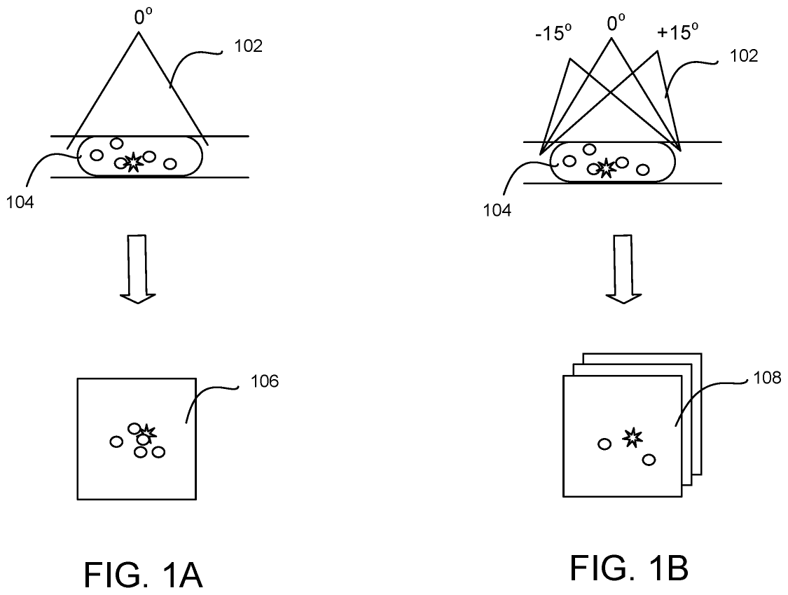

Mammography may be used to capture pictures of a breast with different views (e.g., a craniocaudal (CC) view and/or a mediolateral oblique (MLO) view). As such, a standard mammogram may include four pictures, e.g., a left CC (LCC), a left MLO (LMLO), a right CC (RCC), and a right MLO (RMLO). FIGS. 1A and 1B illustrate examples of mammography techniques, with FIG. 1A showing an example of full-field digital mammography (FFDM) and FIG. 1B showing an example of digital breast tomosynthesis (DBT). As shown in FIG. 1A, FFDM may be considered a 2D imaging modality that may involve passing a burst of X-rays 102 through a compressed breast 104 at a certain angel (e.g., perpendicular to the breast), capturing the X-rays 102 on the opposite side (e.g., using a solid-state detector), and producing a 2D image 106 of the breast based on the captured signals (e.g., the captured X-rays 102 may be converted to electronic signals, which may then be used to generate the 2D image 106). In contrast, the DBT technique shown in FIG. 1B may achieve or resemble the quality of a 3D imagining modality (e.g., DBT may be considered a pseudo 3D imaging modality). As shown, the DBT technique may involve passing the burst of X-rays 102 through the compressed breast 104 at different angles (e.g., 0°, +15°, −15°, etc.) during a scan, acquiring one or more X-ray images of the breast at each of the angles, and reconstructing the individual X-ray images into a series of slices 108 (e.g., thin, high-resolution slice images) that may be displayed individually or as a movie (e.g., in a dynamic cine mode). As such, different from the example FFDM technique shown in FIG. 1A (e.g., which may project the breast 104 from only one angle), the example DBT technique shown in FIG. 1B may project the breast from multiple angles and reconstruct the data collected from those different angles into multiple slice images 108 (e.g., multi-slice data) in which the normal breast tissues (e.g., represented by circles in FIG. 1B) may be clearly distinguished from the lesion (e.g., represented by a star in FIG. 1B). This technique may reduce or eliminate certain problems caused by 2D mammography imaging (e.g., the FFDM technique described herein), resulting in improved diagnostic and screening accuracy.

It should be noted that although FIG. 1B shows only three angles at which x-ray images of the breast 104 are taken, those skilled in the art will appreciate that more angles may be used and more images may be taken during a practical DBT procedure. For example, 15 images of the breast may be taken in an arc from the top and the side of the breast, which may then be reconstructed into multiple non-overlapping slices through the breast. Those skilled in the art will also appreciate that, although not shown in FIG. 1B, a DBT scan may include different views of each breast including, for example, LCC, LMLO, RCC, and RMLO.

The mammography technologies described herein (e.g., DBT and/or FFDM) may provide rich information about the heath state of a breast (e.g., such as the prospect of breast cancer), but the data generated during a mammogram procedure such as a DBT procedure may be voluminous (e.g., 40 to 80 slices per view per breast), posing challenges for human-based data processing. Hence, in embodiments of the present disclosure, machine learning (ML) such as deep learning (DL) techniques may be employed to dissect, analyze, and/or summarize mammography data (e.g., DBT slice images, FFDM images, etc.), and detect the presence of abnormalities (e.g., lesions) automatically.

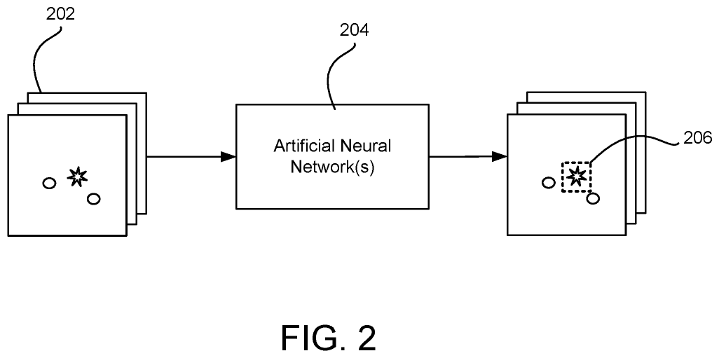

FIG. 2 illustrates an example of using a machine learned detection model to automatically detect an abnormality in a breast based on one or more medical image(s) of the breast. As shown in the figure, a system or apparatus configured to perform the detection task may obtain one or more medical images 202 (e.g., multiple DBT slices or FFDM images) of a breast, and process the images 202 through an artificial neural network (ANN) 204 (e.g., one or more ANNs may be used to accomplish the tasks described herein). The ANN 204 may be used to implement an abnormality detection model, which may be learned (e.g., trained) using an instance of the ANN 204 (e.g., the terms “ANN,” “neural network,” “ML model,” “DL model,” or “artificial intelligence (AI) model” may be used interchangeably in the present disclosure). The medical image(s) 202 may include information indicating the locations, geometries, and/or structures of normal breast tissues (e.g., indicated by the circles in the figures) and/or abnormalities such as a lesion (e.g., indicated by the star in the figure), and the ANN 204 may be trained to distinguish the lesion from the normal breast tissues based on image features associated with the lesion that the ANN may have learned through training. The ANN 204 may indicate the detection of the lesion in various manners. For instance, the ANN 204 may indicate the detection by drawing a bounding shape 206 (e.g., a bounding box or circle) around the lesion (e.g., the area containing the lesion) in the corresponding medical image(s). As another example, the ANN 204 may indicate the detection by setting a probability score that may indicate the likelihood of breast cancer to a specific value (e.g., in the range of 0-1, with 1 indicating the highest likelihood and 0 indicating the lowest likelihood). As yet another example, the ANN 204 may indicate the detection by segmenting the lesion from the corresponding medical image 202. It should be noted here that even though the term “detection” is used in some examples provided herein, the techniques described in this disclosure may also be applied to other tasks including, for example, classification tasks and/or segmentation tasks.

The ML model implemented by the ANN 204 may utilize various architectures including, for example, a one stage architecture (e.g., such as You Only Look Once (YOLO)), a two stage architecture (e.g., such as Faster Region-based Convolutional Neural Network (Faster-RCNN)), an anchor free architecture (e.g., such as Fully Convolutional One-Stage object detection (FCOS)), a transformer-based architecture (e.g., such as Detection Transformer (DETR)), and/or the like. In examples, the ANN 204 may include a plurality of layers such as one or more convolution layers, one or more pooling layers, and/or one or more fully connected layers. Each of the convolution layers may include a plurality of convolution kernels or filters configured to extract features from an input image (e.g., the medical image(s) 202). The convolution operations may be followed by batch normalization and/or linear (or non-linear) activation, and the features extracted by the convolution layers may be down-sampled through the pooling layers and/or the fully connected layers to reduce the redundancy and/or dimension of the features, so as to obtain a representation of the down-sampled features (e.g., in the form of a feature vector or feature map). In some examples (e.g., such as those associated with a segmentation task), the ANN 204 may further include one or more un-pooling layers and one or more transposed convolution layers that may be configured to up-sample and de-convolve the features extracted through the operations described above. As a result of the up-sampling and de-convolution, a dense feature representation (e.g., a dense feature map) of the input image may be derived, and the ANN 204 may be trained (e.g., parameters of the ANN may be adjusted) to predict the presence or non-presence of an abnormality (e.g., lesion) in the input image based on the feature representation. As will be described in greater detail below, the training of the ANN 204 may be conducted using a training dataset generated from unlabeled data, and the parameters of the ANN 204 (e.g., the ML model implemented by the ANN) may be adjusted (e.g., learned) based on various loss functions.

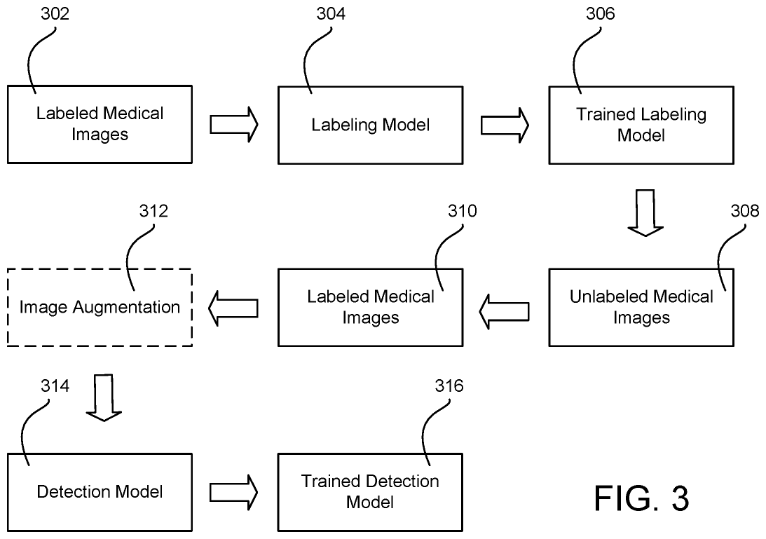

FIG. 3 illustrates example operations that may be associated with training the neural network or ML model described herein for processing mammography images such as DBT and/or FFDM images. As discussed above, conventional neural network training methods may require a large amount of labeled (e.g., annotated) data, which may be difficult to obtain for the purposes described herein (e.g., DBT data may be voluminous and dynamic, posing even more challenges for manual labeling or annotation). Hence, in the example operations illustrated by FIG. 3, the concerned neural network or ML model may be trained using automatically (or semi-automatically) labeled data (e.g., images), for example, in a multi-stage process. For instance, at 302, a first training dataset comprising labeled breast images (e.g., DBT or FFDM images) may be obtained and used to train a labeling model (e.g., an abnormality labeling model) at 304. When referred to herein, labeled or annotated images may include images for which ground truth regarding the existence or non-existence of an abnormality (e.g., lesion) is available (e.g., in the form of a bounding box around the abnormality, a probability score indicating the likelihood of the abnormality, a segmentation mask over the abnormal area, etc.), while unlabeled or un-annotated images may include images for which ground truth regarding the existence or non-existence of an abnormality is not available. The abnormality labeling model 306 trained using the labeled medical images 302 may be capable of predicting (e.g., detecting, classifying, and/or segmenting) an abnormal area (e.g., a lesion) in an unlabeled medical images and annotating (e.g., labeling) the unlabeled medical images based on the prediction. The annotation may be provided, for example, in the form of a markup around the predicted abnormal area (e.g., by drawing a bounding box or circle around the predicted abnormal area) in the otherwise unlabeled medical image.

Given the limited availability of labeled mammogram data, the first training dataset used at 302 may be small, and the abnormality labeling model 306 trained using the first training dataset may not be sufficiently accurate or robust for predicting breast diseases in compliance with clinical requirements. The abnormality labeling model 306 may, however, be used to annotate unlabeled medical images 308 such that a second training dataset 310 comprising the annotated medical images may be obtained. Since unlabeled medical images 308 may be more abundant, the techniques described herein may allow for generation of more labelled training data, which may then be used to train an abnormality detection model at 314 as described herein.

In some examples, all or a subset of the medical images 310 annotated using the abnormality labeling model 306 may be augmented at 312 (e.g., as a post-processing step to the labeling operation described herein) before being used to train the detection model at 314. In other examples, the labeled medical images 310 may be used to train the detection model at 314 without augmentation (e.g., the dotted line in the figure is used to indicate that the augmentation operation may or may not be performed, and that it may be performed for all or a subset of the labeled medical images 310). If performed at 312, the augmentation may include, for example, transforming a labeled medical image 310 with respect to at least one of an image property (e.g., intensity and/or contrast), a geometry, or a feature space of the medical image. The image property related transformation may be accomplished, for example, by manipulating the intensity and/or contrast values of the medical image, the geometric transformation may be accomplished, for example, by rotating, flipping, cropping, or translating the medical image, and the feature space transformation may be accomplished, for example, by adding noise to the medical image or interpolating/extrapolating certain features of the medical images. In examples, the image augmentation operation at 312 may also include mixing two or more of the medical images 310 (e.g., by averaging the medical images) or randomly erasing certain patches from a medical image 310. Through these operations, variations may be added to the second training dataset to improve the adaptability and/or robustness of the detection model trained at 314.

In examples, the augmentation operation at 312 may include removing redundant or overlapped labeling (e.g., using a non-maximum suppression (NMS) technique) from a medical image 310. The augmentation operation at 312 may also include determining that a medical image 310 labeled by the labeling model 306 may be associated with a low confidence score (e.g., below a certain threshold value), and excluding such a medical image from the second training dataset (e.g., the confidence score may be generated as an output of the abnormality labeling model 306). The augmentation operation at 312 may also include masking a labeled medical image 310 (e.g., by revealing only the labeled area and hiding the rest of the image), and adding the masked image to the second training dataset.

One or more of the augmentation operations described herein may also be applied to the annotation or label (e.g., markup) created by the labeling model 306. For example, a bounding box (or other bounding shapes) created by the labeling model 306 may also be transformed with respect to at least one of the intensity, contrast, or geometry of the bounding box, e.g., in a similar manner as for the corresponding medical image itself. This way, even after the transformation, the labeled medical image may still be used to train the detection model at 314.

It should be noted here that the detection model 316 obtained based on the auto-labeled and/or augmented training images may be a different or separate model from the labeling model 306, or the detection model 316 may be a model refined based on the labeling model 306 (e.g., the labeling model 306 may be fine-tuned based on the auto-generated and/or augmented training images to obtain the detection model 316). It should also be noted that the originally labeled medical images 302 may also be used (e.g., in addition to the auto-generated and/or augmented training images) to train the detection model 316.

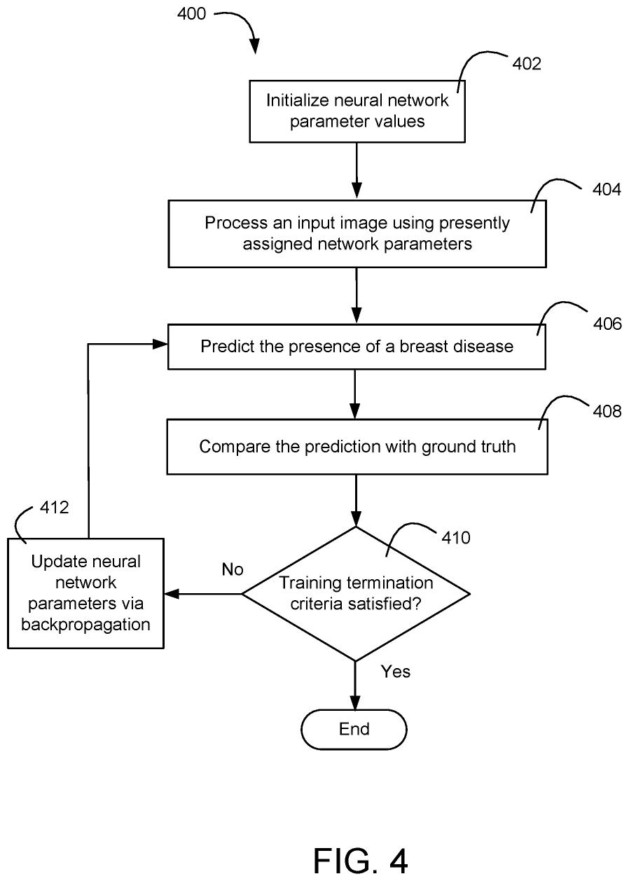

FIG. 4 illustrates an example process 400 for training a neural network (e.g., the ANN 204 of FIG. 2 or the ML model implemented by the ANN) to perform one or more of the tasks described herein. As shown, the training process 400 may include initializing the operating parameters of the neural network (e.g., weights associated with various layers of the neural network) at 402, for example, by sampling from a probability distribution or by copying the parameters of another neural network having a similar structure. The training process 400 may further include processing an input image (e.g., a manually label training image or automatically labelled/augmented training image as described herein) using presently assigned parameters of the neural network at 404, and making a prediction about the presence of a breast abnormality (e.g., a lesion) at 406. The prediction result may be compared, at 408, to a ground truth (e.g., the label or annotation described herein) that may indicate a true status of the breast abnormality (e.g., whether the abnormality truly exists and/or the location of the abnormality). As a result of the comparison, a loss associated with the prediction may be determined, e.g., based on a loss function such as a mean squared error between the prediction result and the ground truth, an L1 norm, an L2 norm, etc. Then, at 410, the loss may be used to determine whether one or more training termination criteria are satisfied. For example, the training termination criteria may be determined to be satisfied if the loss is below a threshold value or if the change in the loss between two training iterations falls below a threshold value. If the determination at 410 is that the termination criteria are satisfied, the training may end; otherwise, the presently assigned network parameters may be adjusted at 412, for example, by backpropagating a gradient descent of the loss function through the network before the training returns to 406.

For simplicity of explanation, the training steps are depicted and described herein with a specific order. It should be appreciated, however, that the training operations may occur in various orders, concurrently, and/or with other operations not presented or described herein. Furthermore, it should be noted that not all operations that may be included in the training method are depicted and described herein, and not all illustrated operations are required to be performed.

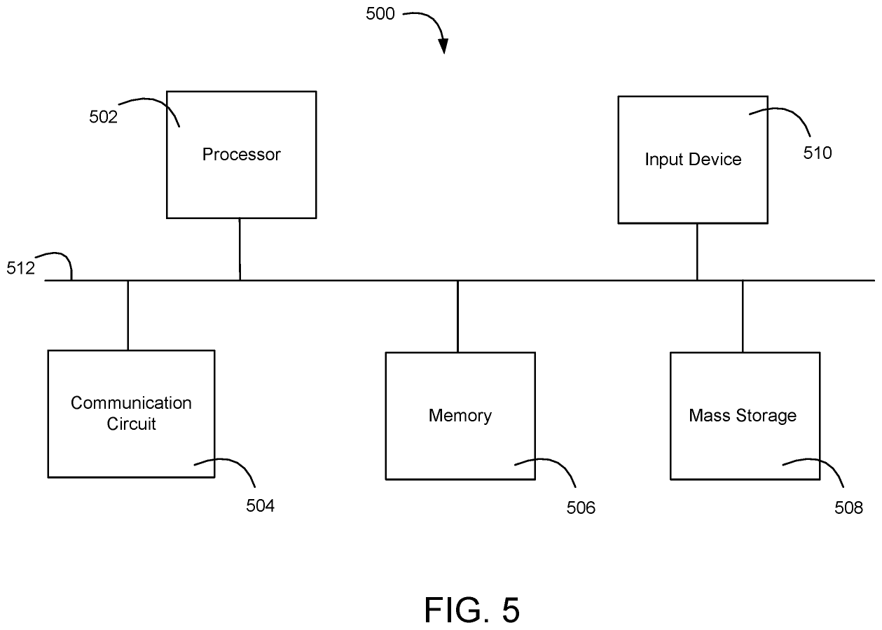

The systems, methods, and/or instrumentalities described herein may be implemented using one or more processors, one or more storage devices, and/or other suitable accessory devices such as display devices, communication devices, input/output devices, etc. FIG. 5 illustrates an example apparatus 500 that may be configured to perform the tasks described herein. As shown, apparatus 500 may include a processor (e.g., one or more processors) 502, which may be a central processing unit (CPU), a graphics processing unit (GPU), a microcontroller, a reduced instruction set computer (RISC) processor, application specific integrated circuits (ASICs), an application-specific instruction-set processor (ASIP), a physics processing unit (PPU), a digital signal processor (DSP), a field programmable gate array (FPGA), or any other circuit or processor capable of executing the functions described herein. Apparatus 500 may further include a communication circuit 504, a memory 506, a mass storage device 508, an input device 510, and/or a communication link 512 (e.g., a communication bus) over which the one or more components shown in the figure may exchange information.

Communication circuit 504 may be configured to transmit and receive information utilizing one or more communication protocols (e.g., TCP/IP) and one or more communication networks including a local area network (LAN), a wide area network (WAN), the Internet, a wireless data network (e.g., a Wi-Fi, 3G, 4G/LTE, or 5G network). Memory 506 may include a storage medium (e.g., a non-transitory storage medium) configured to store machine-readable instructions that, when executed, cause processor 502 to perform one or more of the functions described herein. Examples of the machine-readable medium may include volatile or non-volatile memory including but not limited to semiconductor memory (e.g., electrically programmable read-only memory (EPROM), electrically erasable programmable read-only memory (EEPROM)), flash memory, and/or the like. Mass storage device 508 may include one or more magnetic disks such as one or more internal hard disks, one or more removable disks, one or more magneto-optical disks, one or more CD-ROM or DVD-ROM disks, etc., on which instructions and/or data may be stored to facilitate the operation of processor 502. Input device 510 may include a keyboard, a mouse, a voice-controlled input device, a touch sensitive input device (e.g., a touch screen), and/or the like for receiving user inputs to apparatus 500.

It should be noted that apparatus 500 may operate as a standalone device or may be connected (e.g., networked or clustered) with other computation devices to perform the tasks described herein. And even though only one instance of each component is shown in FIG. 5, a skilled person in the art will understand that apparatus 500 may include multiple instances of one or more of the components shown in the figure.

While this disclosure has been described in terms of certain embodiments and generally associated methods, alterations and permutations of the embodiments and methods will be apparent to those skilled in the art. Accordingly, the above description of example embodiments does not constrain this disclosure. Other changes, substitutions, and alterations are also possible without departing from the spirit and scope of this disclosure. In addition, unless specifically stated otherwise, discussions utilizing terms such as “analyzing,” “determining,” “enabling,” “identifying,” “modifying” or the like, refer to the actions and processes of a computer system, or similar electronic computing device, that manipulates and transforms data represented as physical (e.g., electronic) quantities within the computer system's registers and memories into other data represented as physical quantities within the computer system memories or other such information storage, transmission or display devices.

It is to be understood that the above description is intended to be illustrative, and not restrictive. Many other implementations will be apparent to those of skill in the art upon reading and understanding the above description.

What is claimed is:

1. An apparatus, comprising:
at least one processor configured to:
obtain a medical image of a breast;
determine, based on the medical image, whether an abnormality exists in the breast, wherein the determination is made based on a machine-learned abnormality detection model, and wherein the abnormality detection model is learned through a process that comprises:
training an abnormality labeling model based on a first training dataset comprising labeled medical images;
deriving a second training dataset based on unlabeled medical images, wherein the derivation comprises annotating the unlabeled medical images based on the trained abnormality labeling model; and
training the abnormality detection model based at least on the second training dataset; and
indicate a result of the determination.

2. The apparatus of claim 1, wherein the medical image includes a digital breast tomosynthesis image or a full-field digital mammography image.

3. The apparatus of claim 1, wherein the abnormality labeling model is trained to predict an abnormal area in each of the unlabeled medical images and annotate the each of the unlabeled medical images based on the prediction.

4. The apparatus of claim 3, wherein annotating the each of the unlabeled medical images based on the prediction comprising marking the predicted abnormal area in the each of the unlabeled medical images.

5. The apparatus of claim 1, wherein the derivation of the second training dataset further comprises transforming one or more medical images annotated by the abnormality labeling model with respect to at least one of an intensity or a geometry of each of the one or more medical images.

6. The apparatus of claim 5, wherein each of the one or more medical images annotated by the abnormality labeling model comprises a markup around a predicted abnormal area, and wherein the derivation of the second training dataset further comprises transforming at least one of an intensity or a geometry of the markup.

7. The apparatus of claim 1, wherein the derivation of the second training dataset further comprises masking one or more medical images annotated by the abnormality labeling model.

8. The apparatus of claim 1, wherein the derivation of the second training dataset further comprises removing a redundant prediction of an abnormal area in a medical image annotated by the abnormality labeling model.

9. The apparatus of claim 1, wherein the derivation of the second training dataset further comprises determining that a medical image annotated by the abnormality labeling model is associated with a confidence score below a threshold value, and excluding the medical image from the second training dataset based on the determination.

10. The apparatus of claim 1, wherein the abnormality detection model is trained as a different model than the abnormality labeling model, or as refinement of the abnormality labeling model.

11. The apparatus of claim 1, wherein the at least one processor being configured to indicate the result of the determination comprises the at least one processor being configured to mark the abnormality in the medical image.

12. A method of processing medical images, the method comprising:
obtaining a medical image of a breast;
determining, based on the medical image, whether an abnormality exists in the breast, wherein the determination is made based on a machine-learned abnormality detection model, and wherein the abnormality detection model is learned through a process that comprises:
training an abnormality labeling model based on a first training dataset comprising labeled medical images;
deriving a second training dataset based on unlabeled medical images, wherein the derivation comprises annotating the unlabeled medical images based on the trained abnormality labeling model; and
training the abnormality detection model based at least on the second training dataset; and
indicating a result of the determination.

13. The method of claim 12, wherein the medical image includes a digital breast tomosynthesis (DBT) image or a full-field digital mammography (FFDM) image.

14. The method of claim 12, wherein the abnormality labeling model is trained to predict an abnormal area in each of the unlabeled medical images and annotate the each of the unlabeled medical images based on the prediction.

15. The method of claim 14, wherein annotating the each of the unlabeled medical images based on the prediction comprising marking the predicted abnormal area in the each of the unlabeled medical images.

16. The method of claim 12, wherein the derivation of the second training dataset further comprises transforming one or more medical images annotated by the abnormality labeling model with respect to at least one of an intensity or a geometry of each of the one or more medical images.

17. The method of claim 16, wherein each of the one or more medical images annotated by the abnormality labeling model comprises a markup around a predicted abnormal area, and wherein the derivation of the second training dataset further comprises transforming at least one of an intensity or a geometry of the markup.

18. The method of claim 12, wherein the derivation of the second training dataset further comprises masking one or more medical images annotated by the abnormality labeling model or removing a redundant prediction of an abnormal area in a medical image annotated by the abnormality labeling model.

19. The method of claim 12, wherein the derivation of the second training dataset further comprises determining that a medical image annotated by the abnormality labeling model is associated with a confidence score below a threshold value, and excluding the medical image from the second training dataset based on the determination.

20. A method of training a neural network for processing medical images generated based on digital breast tomosynthesis (DBT) or full-field digital mammography (FFDM), the method comprising:
training an abnormality labeling model based on a first training dataset comprising labeled DBT or FFDM images;
deriving a second training dataset based on unlabeled DBT or FFDM images, wherein the derivation comprises predicting an abnormal area in each of the unlabeled DBT or FFDM images, and annotating the predicted abnormal area in the each of the unlabeled DBT or FFDM images; and
training the abnormality detection model based at least on the second training dataset.
